(19) United States
(12) Patent Application Publication
(10) Pub. No.: US 2024/0000431 A1
(43) Pub. Date: Jan. 4, 2024

(54) ULTRASOUND SYSTEM AND CONTROL METHOD OF ULTRASOUND SYSTEM

(71) Applicant: FUJIFILM Corporation, Tokyo (JP)

(72) Inventor: Hiroyuki KARASAWA, Kanagawa (JP)

(73) Assignee: FUJIFILM Corporation, Tokyo (JP)

(21) Appl. No.: 18/468,221

(22) Filed: Sep. 15, 2023

Related U.S. Application Data
(63) Continuation of application No. PCT/JP2022/005839, filed on Feb. 15, 2022.

(30) Foreign Application Priority Data
Mar. 24, 2021 (JP) 2021-049449

(51) Int. Cl. A61B 8/00; A61B 8/14; A61B 8/08

(52) U.S. Cl. CPC A61B 8/463; A61B 8/467; A61B 8/4483; A61B 8/145; A61B 8/5207; A61B 8/4472; A61B 8/5246; A61B 8/56; A61B 8/488

(57) ABSTRACT

An ultrasound system includes an ultrasound probe, a user interface device (3) connected to the ultrasound probe by wire, and a mobile information terminal connected to at least one of the ultrasound probe or the user interface device (3), in which the ultrasound probe includes a transducer array, a transmission/reception circuit that generates a sound ray signal based on a reception signal acquired by the transducer array, and a first image generation unit that generates a first ultrasound image based on the sound ray signal, the user interface device (3) includes a second image generation unit (34) that generates a second ultrasound image based on the sound ray signal, and the mobile information terminal includes a monitor that displays the first ultrasound image or the second ultrasound image.

### CROSS-REFERENCE TO RELATED APPLICATIONS

This application is a Continuation of PCT International Application No. PCT/JP2022/005839 filed on Feb. 15, 2022, which claims priority under 35 U.S.C. § 119(a) to Japanese Patent Application No. 2021-049449 filed on Mar. 24, 2021. The above applications are hereby expressly incorporated by reference, in their entirety, into the present application.

### BACKGROUND OF THE INVENTION

### 1. Field of the Invention

The present invention relates to an ultrasound system comprising a user interface device used for an input operation of a user and a control method of an ultrasound system.

### 2. Description of the Related Art

In the related art, in the medical field, an ultrasound system that examines an inside of a subject by utilizing an ultrasound image has been put into practical use. In recent years, for the purpose of being used in the field of home medical care or the like, for example, as disclosed in JP2010-528696A, an ultrasound system has been developed that allows a user to easily carry and easily perform an examination of a subject.

The ultrasound system disclosed in JP2010-528696A comprises an ultrasound probe, and a user interface device that is connected to the ultrasound probe by wire and allows the user can easily perform an input operation. The user interface device is connected to an external apparatus, such as an ultrasound diagnostic apparatus having a monitor, in which an ultrasound image generated by the ultrasound probe is displayed.

### SUMMARY OF THE INVENTION

With the ultrasound system disclosed in JP2010-528696A, the user can easily perform an ultrasound examination with respect to the subject. However, in general, in the ultrasound probe, it is necessary to suppress heat generation in the ultrasound probe in order to reduce a burden on the subject or the like, and thus it is difficult to mount a circuit on an ultrasound probe in which a heat generation amount is increased due to complicated processing. Further, in the ultrasound probe capable of wireless communication as disclosed in JP2010-528696A, since power is supplied from a battery provided inside the ultrasound probe, in a case of considering that the examination with respect to the subject is performed in a sufficiently stable manner, it is difficult to mount a circuit that performs processing with large power consumption in the ultrasound probe.

Therefore, in the ultrasound system disclosed in JP2010-528696A, it is difficult for the user to perform various examinations with respect to the subject, for example, to perform the examination with respect to the subject by generating a plurality of types of ultrasound images or the like.

The present invention has been made to solve such a problem in the related art, and is to provide an ultrasound system that allows a user to easily perform various examinations with respect to a subject and a control method of an ultrasound system.

In order to achieve the above object, an aspect of the present invention relates to an ultrasound system comprising an ultrasound probe, a user interface device connected to the ultrasound probe by wire, and a mobile information terminal connected to at least one of the ultrasound probe or the user interface device, in which the ultrasound probe includes a transducer array, a transmission/reception circuit that transmits an ultrasound wave from the transducer array and generates a sound ray signal based on a reception signal acquired by the transducer array, and a first image generation unit that generates a first ultrasound image based on the sound ray signal generated by the transmission/reception circuit, the user interface device includes a second image generation unit that generates a second ultrasound image based on the sound ray signal generated by the transmission/reception circuit of the ultrasound probe, and the mobile information terminal includes a monitor that displays the first ultrasound image generated by the first image generation unit of the ultrasound probe or the second ultrasound image generated by the second image generation unit of the user interface device.

The second ultrasound image generated by the second image generation unit of the user interface device may include at least one of an ultrasound elastography image, a low-speed blood flow image, a high-sensitivity low-speed image, or a contrast image.

The mobile information terminal may include an input device that allows a user to perform an input operation in a case in which the mobile information terminal is not connected to the user interface device.

In addition, the mobile information terminal may be wirelessly connected to the ultrasound probe, and the first ultrasound image generated by the first image generation unit of the ultrasound probe may be wirelessly transmitted from the ultrasound probe to the mobile information terminal and displayed on the monitor.

The user interface device may include a power circuit for supplying power to the ultrasound probe connected to the user interface device by wire.

The power circuit of the user interface device may supply a transmission voltage for transmitting the ultrasound wave from the transducer array of the ultrasound probe, to the transmission/reception circuit.

Each of a plurality of the ultrasound probes may be connected to the user interface device by wire, the user interface device may include a probe selection unit that selects one ultrasound probe among the plurality of ultrasound probes, and the second image generation unit of the user interface device may generate the second ultrasound image based on the sound ray signal generated by the transmission/reception circuit of the ultrasound probe selected by the probe selection unit.

The user interface device may include a continuous wave Doppler circuit that drives the transducer array in a continuous wave Doppler mode and generates a sound ray signal based on the reception signal acquired by the transducer array in a case in which the ultrasound probe is able to operate in the continuous wave Doppler mode, and the second image generation unit may generate a continuous wave Doppler image based on the sound ray signal generated by the continuous wave Doppler circuit.

Another aspect of the present invention relates to a control method of an ultrasound system including an ultrasound probe, a user interface device connected to the ultrasound probe by wire, and a mobile information terminal connected to at least one of the ultrasound probe or the user interface device, the control method comprising causing the ultrasound probe to transmit an ultrasound wave from a transducer array, to generate a sound ray signal based on a reception signal acquired by the transducer array, and to generate a first ultrasound image based on the sound ray signal, causing the user interface device to generate a second ultrasound image based on the sound ray signal, and causing the mobile information terminal to display the first ultrasound image or the second ultrasound image on a monitor.

According to the aspect of the present invention, the ultrasound system comprises an ultrasound probe, a user interface device connected to the ultrasound probe by wire, and a mobile information terminal connected to at least one of the ultrasound probe or the user interface device, the ultrasound probe includes a transducer array, a transmission/reception circuit that transmits an ultrasound wave from the transducer array and generates a sound ray signal based on a reception signal acquired by the transducer array, and a first image generation unit that generates a first ultrasound image based on the sound ray signal generated by the transmission/reception circuit, the user interface device includes a second image generation unit that generates a second ultrasound image based on the sound ray signal generated by the transmission/reception circuit of the ultrasound probe, and the mobile information terminal includes a monitor that displays the first ultrasound image generated by the first image generation unit of the ultrasound probe or the second ultrasound image generated by the second image generation unit of the user interface device. Therefore, the user can easily perform various examinations with respect to the subject.

### BRIEF DESCRIPTION OF THE DRAWINGS

FIG. 1 is a block diagram showing a configuration of an ultrasound system according to a first embodiment of the present invention.

FIG. 2 is a block diagram showing a configuration of an ultrasound probe in the first embodiment of the present invention.

FIG. 3 is a block diagram showing a configuration of a reception circuit in the first embodiment of the present invention.

FIG. 4 is a block diagram showing a configuration of a user interface device in the first embodiment of the present invention.

FIG. 5 is a block diagram showing a configuration of a mobile information terminal in the first embodiment of the present invention.

FIG. 6 is a block diagram showing a configuration of an ultrasound probe in a second embodiment of the present invention.

FIG. 7 is a block diagram showing a configuration of a user interface device in the second embodiment of the present invention.

FIG. 8 is a block diagram showing a configuration of a user interface device in a third embodiment of the present invention.

FIG. 9 is a block diagram showing a configuration of a user interface device in a fourth embodiment of the present invention.

### DESCRIPTION OF THE PREFERRED EMBODIMENTS

Hereinafter, embodiments of the present invention will be described based on the accompanying drawings.

### First Embodiment

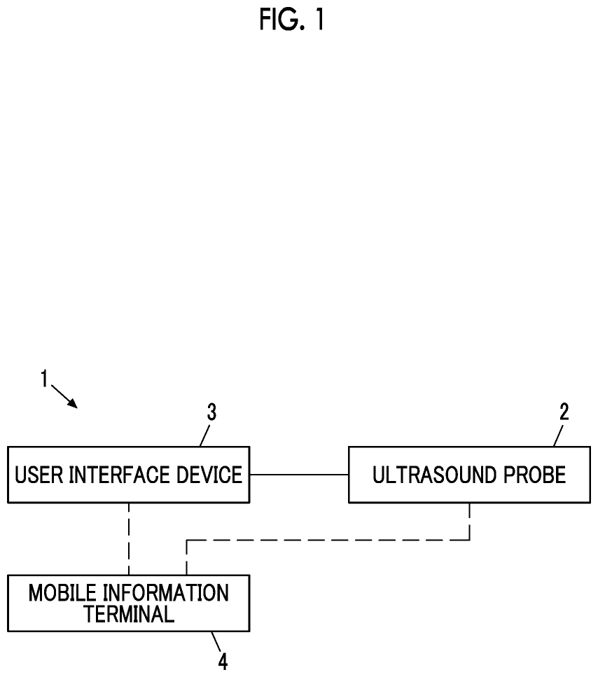

FIG. 1 shows a configuration of an ultrasound system 1 according to the first embodiment of the present invention. As shown in FIG. 1, the ultrasound system 1 comprises an ultrasound probe 2, and a user interface device 3 is connected to the ultrasound probe 2 by wire. In addition, a mobile information terminal 4 is wirelessly connected to the ultrasound probe 2 and the user interface device 3.

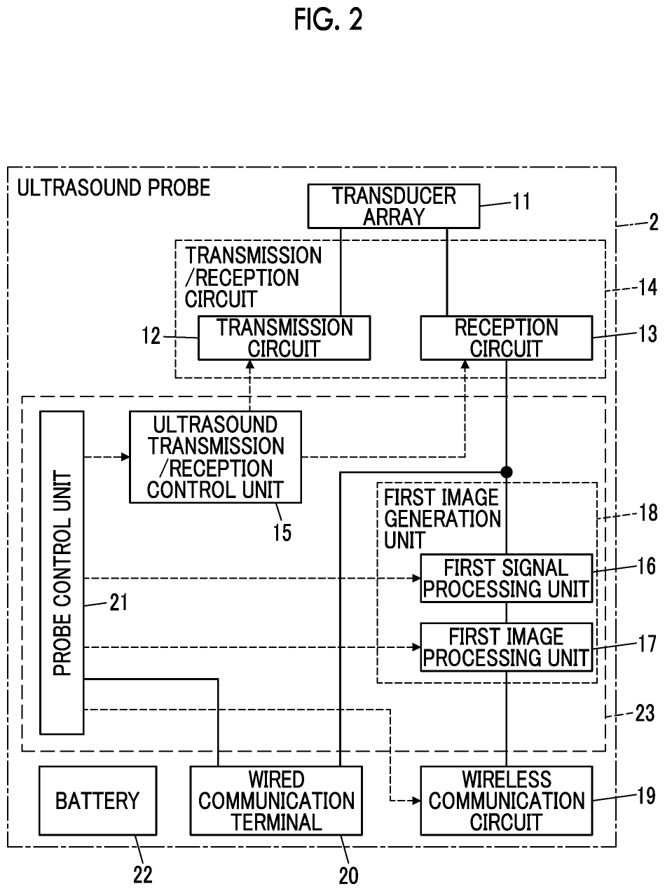

As shown in FIG. 2, the ultrasound probe 2 comprises a transducer array 11, and a transmission circuit 12 and a reception circuit 13 are connected to the transducer array 11. A transmission/reception circuit 14 is configured by using the transmission circuit 12 and the reception circuit 13. In addition, an ultrasound transmission/reception control unit 15 is connected to the transmission circuit 12 and the reception circuit 13. In addition, a first signal processing unit 16 and a first image processing unit 17 are sequentially connected to the reception circuit 13. A first image generation unit 18 is configured by using the first signal processing unit 16 and the first image processing unit 17. In addition, a wireless communication circuit 19 is connected to the first image processing unit 17. In addition, a wired communication terminal 20 is connected to the reception circuit 13.

In addition, a probe control unit 21 is connected to the ultrasound transmission/reception control unit 15, the first signal processing unit 16, the first image processing unit 17, the wireless communication circuit 19, and the wired communication terminal 20.

In addition, an ultrasound probe processor 23 is configured by using the ultrasound transmission/reception control unit 15, the first signal processing unit 16, the first image processing unit 17, and the probe control unit 21.

Also, a battery 22 is built in the ultrasound probe 2.

The transducer array 11 of the ultrasound probe 2 includes a plurality of ultrasound transducers arranged one-dimensionally or two-dimensionally. Each of these transducers transmits an ultrasound wave according to a drive voltage number supplied from the transmission circuit 12 and receives a reflected wave from a subject to output a reception signal. For example, each transducer is configured by using an element in which electrodes are formed at both ends of a piezoelectric body consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

The transmission circuit 12 includes, for example, a plurality of pulse generators, and supplies each of drive signals to the plurality of transducers by adjusting a delay amount such that the ultrasound waves transmitted from the plurality of transducers of the transducer array 11 form an ultrasound beam based on a transmission delay pattern selected in response to a control signal from the ultrasound transmission/reception control unit 15. Thus, in a case in which a pulsed or continuous wave voltage is applied to the electrodes of the transducers of the transducer array 11, the piezoelectric body expands and contracts to generate a pulsed or continuous wave ultrasound wave from each transducer, and the ultrasound beam is formed from the combined wave of these ultrasound waves.

The transmitted ultrasound beam is reflected by, for example, a target, such as a part in the subject, and propagates as a so-called ultrasound echo toward the transducer array 11. The ultrasound echo propagating toward the transducer array 11 in this way is received by each of the ultrasound transducers that constitute the transducer array 11. In this case, each of the ultrasound transducers constituting the transducer array 11 expands and contracts by receiving the propagating ultrasound echo to generate electric signals, and outputs a reception signal which is these electric signals to the reception circuit 13.

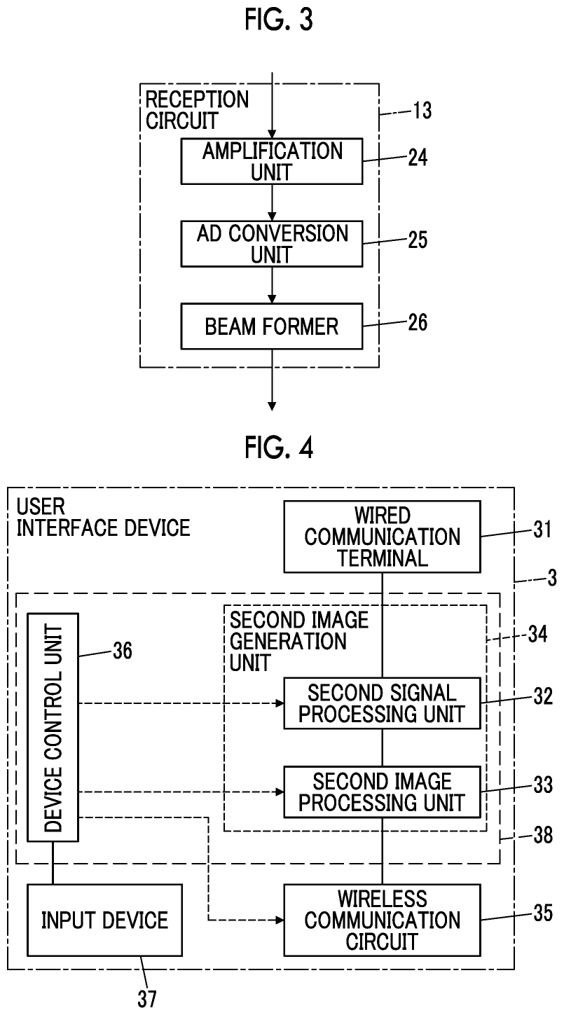

The reception circuit 13 performs processing of the reception signal output from the transducer array 11 in response to the control signal from the ultrasound transmission/reception control unit 15. As shown in FIG. 3, the reception circuit 13 has a configuration in which an amplification unit 24, an analog digital (AD) conversion unit 25, and a beam former 26 are connected in series. The amplification unit 24 amplifies the reception signal input from each of the transducers that constitute the transducer array 11, and transmits the amplified reception signal to the AD conversion unit 25. The AD conversion unit 25 converts the reception signal transmitted from the amplification unit 24 into digitized element data, and sends out these element data to the beam former 26. The beam former 26 performs reception focus processing of performing addition (phasing addition) by giving the delay to each of the element data according to a set sound speed, based on a reception delay pattern selected in response to the control signal from the ultrasound transmission/reception control unit 15. By performing this reception focus processing, a sound ray signal in which a focus of the ultrasound echo is narrowed down is generated.

Under the control of the probe control unit 21, the ultrasound transmission/reception control unit 15 controls the transmission circuit 12 and the reception circuit 13 to perform the transmission of the ultrasound beam and the reception of the ultrasound echo. The ultrasound transmission/reception control unit 15 can perform, for example, the selection of the ultrasound transducer used for transmitting the ultrasound wave, the selection of the ultrasound transducer used for receiving the ultrasound wave, and the control of timings of the transmission and the reception of the ultrasound wave.

The wired communication terminal 20 is connected to a cable (not shown) connected to the user interface device 3, and is a terminal for transmitting the sound ray signal generated by the reception circuit 13 to the user interface device 3 via the cable. Any known terminal for wired communication can be used as the wired communication terminal 20.

The first signal processing unit 16 performs envelope detection processing after performing, with respect to the sound ray signal generated by the beam former 26 of the reception circuit 13, correction of the attenuation due to a propagation distance according to a depth of a position at which the ultrasound wave is reflected, and generates a signal that is tomographic image information related to a tissue in the subject.

The first image processing unit 17 generates a first ultrasound image by performing raster conversion on the signal generated by the first signal processing unit 16 into an image signal according to a scanning method of a normal television signal, and performing various necessary image processing, such as brightness correction, gradation correction, sharpness correction, and color correction, with respect to the image signal generated in this way. In addition, the first image processing unit 17 compresses the first ultrasound image generated in this manner into, for example, a format, such as joint photographic experts group (JPEG), and sends out the first ultrasound image to the wireless communication circuit 19.

Here, as the first ultrasound image, the first image processing unit 17 can generate, for example, a brightness mode (B mode) image, a motion mode (M mode) image, a color Doppler image, or a pulsed Doppler image.

The wireless communication circuit 19 includes an antenna for performing the transmission and the reception of the radio waves, and performs wireless communication with the mobile information terminal 4. The wireless communication circuit 19 transmits, for example, the first ultrasound image generated by the first image processing unit 17 to the mobile information terminal 4 by wireless communication. In this case, the wireless communication circuit 19 modulates the carrier based on the first ultrasound image to generate a transmission signal, and transmits the generated transmission signal to the mobile information terminal 4. As the carrier modulation method, for example, amplitude shift keying (ASK), phase shift keying (PSK), quadrature phase shift keying (QPSK), 16 quadrature amplitude modulation (16QAM), and the like are used.

The probe control unit 21 performs control of each unit of the ultrasound probe 2 based on a program or the like stored in advance.

The battery 22 is built in the ultrasound probe 2, and supplies power to each circuit of the ultrasound probe 2.

It should be noted that the ultrasound probe processor 23 including the ultrasound transmission/reception control unit 15, the first signal processing unit 16, the first image processing unit 17, and the probe control unit 21 is configured by using a central processing unit (CPU) and a control program for causing the CPU to execute various types of processing, but may be configured by using a field programmable gate array (FPGA), a digital signal processor (DSP), an application specific integrated circuit (ASIC), a graphics processing unit (GPU), and another integrated circuit (IC), or may be configured by using a combination thereof.

In addition, the ultrasound transmission/reception control unit 15, the first signal processing unit 16, the first image processing unit 17, and the probe control unit 21 of the ultrasound probe processor 23 can also be configured by being partially or wholly integrated into one CPU or the like.

As shown in FIG. 4, the user interface device 3 comprises a wired communication terminal 31, and a second signal processing unit 32, a second image processing unit 33, and a wireless communication circuit 35 are sequentially connected to the wired communication terminal 31. In addition, a second image generation unit 34 is configured by using the second signal processing unit 32 and the second image processing unit 33.

In addition, a device control unit 36 is connected to the second signal processing unit 32, the second image processing unit 33, and the wireless communication circuit 35. In addition, an input device 37 is connected to the device control unit 36.

In addition, the user interface device processor 38 is configured by using the second signal processing unit 32, the second image processing unit 33, and the device control unit 36.

The wired communication terminal 31 of the user interface device 3 is connected to one end of the cable (not shown) having the other end connected to the wired communication terminal 20 of the ultrasound probe 2, and is a terminal for receiving the sound ray signal transmitted from the reception circuit 13 of the ultrasound probe 2 via the wired communication terminal 20. Any known terminal for wired communication can be used as the wired communication terminal 31.

The second signal processing unit 32 performs quadrature detection processing and the envelope detection processing after performing, with respect to the sound ray signal sent out from the wired communication terminal 31, the correction of the attenuation due to the propagation distance according to the depth of the position at which the ultrasound wave is reflected, and generates the signal that is the tomographic image information related to the tissue in the subject.

The second image processing unit 33 generates a second ultrasound image by performing raster conversion on the signal generated by the second signal processing unit 32 into the image signal according to the scanning method of the normal television signal, and performing various necessary image processing, such as the brightness correction, the gradation correction, the sharpness correction, and the color correction, with respect to the image signal generated in this way. The second image processing unit 33 sends out the second ultrasound image generated in this manner to the wireless communication circuit 35.

Here, the second image processing unit 33 can generate, for example, a so-called ultrasound elastography image, a low-speed blood flow image, a high-sensitivity low-speed image, a contrast image, or the like as the second ultrasound image.

Here, the ultrasound elastography image is an image representing a distribution of the hardness of the tissue by coloring by measuring the sound speed in the tissue of the subject by utilizing the fact that the sound speed propagating in a medium is determined by elasticity of the medium. The ultrasound elastography image applied to the present invention also includes, for example, an ultrasound elastography image generated by using a method called shear wave measurement (SWM).

In addition, the low-speed blood flow image is a Doppler image which images a distribution of the speed of the blood flow to a region in which the speed is lower than a normal Doppler image by eliminating an effect of motion artifacts appearing due to the motion of the tissue other than the blood flow in the blood vessel in so-called Doppler measurement.

In addition, the high-sensitivity low-speed image is an image in which fine blood flow is visualized by using a so-called eFLOW or B-flow coded transmission technique.

In addition, the contrast image is obtained by administering a contrast agent to the subject, analyzing the sound ray signal using a so-called pulse inversion method, and detecting a high harmonic signal from the contrast agent to generate the image based on the high harmonic signal.

As described above, the second image generation unit 34 generates the ultrasound image, which requires processing that has a larger computing amount and is more complicated than the first ultrasound image generated by the first image generation unit 18 in the ultrasound probe 2 in a case of the generation.

The wireless communication circuit 35 of the user interface device 3 includes an antenna for performing the transmission and the reception of the radio waves, and performs wireless communication with the mobile information terminal 4. The wireless communication circuit 35 transmits, for example, the second ultrasound image generated by the second image processing unit 33 to the mobile information terminal 4 by wireless communication. In this case, the wireless communication circuit 35 modulates the carrier based on the second ultrasound image to generate a transmission signal, and transmits the generated transmission signal to the mobile information terminal 4. As the carrier modulation method, for example, the ASK, the PSK, the QPSK, the 16QAM, or the like is used.

The input device 37 includes a dial, a track ball, various buttons, and the like, and allows the user to perform an input operation.

The device control unit 36 performs control of each unit of the user interface device 3 based on a program or the like stored in advance.

It should be noted that the user interface device processor 38 including the second signal processing unit 32, the second image processing unit 33, and the device control unit 36 is configured by using a CPU and a control program for causing the CPU to execute various types of processing, but may be configured by using an FPGA, a DSP, an ASIC, a GPU, and another IC, or may be configured by using a combination thereof.

In addition, the second signal processing unit 32, the second image processing unit 33, and the device control unit 36 of the user interface device processor 38 can also be configured by being partially or wholly integrated into one CPU or the like.

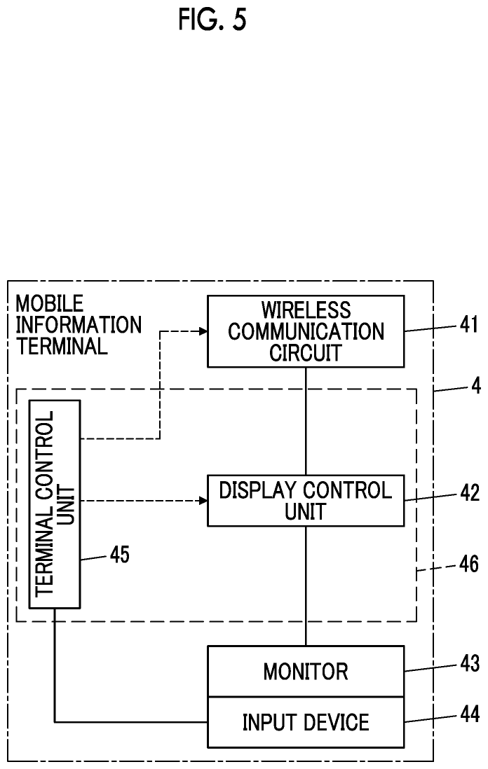

As shown in FIG. 5, the mobile information terminal 4 comprises a wireless communication circuit 41, and a display control unit 42 and a monitor 43 are sequentially connected to the wireless communication circuit 41. In addition, the mobile information terminal 4 comprises an input device 44 including a touch panel, which is disposed to be superimposed on the monitor 43. A terminal control unit 45 is connected to the input device 44. In addition, the terminal control unit 45 is connected to the wireless communication circuit 41 and the display control unit 42.

In addition, a mobile information terminal processor 46 is configured by using the display control unit 42 and the terminal control unit 45.

The wireless communication circuit 41 of the mobile information terminal 4 includes an antenna for performing the transmission and the reception of the radio waves, and performs wireless communication with the wireless communication circuit 19 of the ultrasound probe 2 and the wireless communication circuit 35 of the user interface device 3. Under the control of the terminal control unit 45, the wireless communication circuit 41 receives, for example, the first ultrasound image or the like from the wireless communication circuit 19 of the ultrasound probe 2, and receives the second ultrasound image from the wireless communication circuit 35 of the user interface device 3, information indicating an instruction input from the user via the input device 37, and the like.

In addition, the wireless communication circuit 41 transmits, for example, the information indicating the instruction input from the user via the input device 44 of the mobile information terminal 4 and the like to the wireless communication circuit 19 of the ultrasound probe 2 and the wireless communication circuit 35 of the user interface device 3. The wireless communication circuit 41 modulates the carrier based on the transmitted data to generate a transmission signal in a case of transmitting the data to the wireless communication circuit 19 of the ultrasound probe 2 and the wireless communication circuit 35 of the user interface device 3. As the carrier modulation method, for example, the ASK, the PSK, the QPSK, the 16QAM, or the like is used.

The terminal control unit 45 performs control of each unit of the mobile information terminal 4 according to a program or the like recorded in advance.

Under the control of the terminal control unit 45, the display control unit 42 performs predetermined processing with respect to the first ultrasound image transmitted from the wireless communication circuit 19 of the ultrasound probe 2 and the second ultrasound image transmitted from the wireless communication circuit 35 of the user interface device 3, and displays the first ultrasound image and the second ultrasound image on the monitor 43.

Under the control of the display control unit 42, the monitor 43 performs various displays. The monitor 43 includes, for example, a display device, such as a liquid crystal display (LCD) or an organic electroluminescence display (organic EL display).

The input device 44 allows the user to perform the input operation, includes a so-called touch panel, and is disposed to be superimposed on the monitor 43.

It should be noted that the mobile information terminal processor 46 including the display control unit 42 and the terminal control unit 45 is configured by using a CPU and a control program for causing the CPU to execute various types of processing, but may be configured by using an FPGA, a DSP, an ASIC, a GPU, and another IC, or may be configured by using a combination thereof.

In addition, the display control unit 42 and the terminal control unit 45 of the mobile information terminal processor 46 can also be configured by being partially or wholly integrated into one CPU or the like.

Hereinafter, an operation of the ultrasound system 1 according to first embodiment of the present invention will be described.

First, the operation of the ultrasound system 1 in a case in which the first ultrasound image is generated in the ultrasound probe 2, and the first ultrasound image is displayed on the monitor 43 of the mobile information terminal 4 will be described.

First, the user inputs an instruction to generate the first ultrasound image via the input device 37 of the user interface device 3. In this case, for example, the input device 37 comprises a button for inputting the instruction to generate the first ultrasound image, and the user can press this button to input the instruction to generate the first ultrasound image.

Next, information indicating the instruction to generate the first ultrasound image is transmitted to the wired communication terminal 20 of the ultrasound probe 2 via the wired communication terminal 31 of the user interface device 3, and is transmitted from the wired communication terminal 20 to the probe control unit 21. The probe control unit 21 controls the transmission/reception circuit 14 via the ultrasound transmission/reception control unit 15 to cause the transducer array 11 to perform the transmission and the reception of the ultrasound wave for generating the first ultrasound image.

In a case in which the ultrasound beam is transmitted from the transducer array 11 into the subject, and the ultrasound echo is received by the reception circuit 13, the reception circuit 13 generates the sound ray signal, and sends out the generated sound ray signal to the first signal processing unit 16.

The first signal processing unit 16 performs the envelope detection processing after performing, with respect to the sound ray signal sent out from the reception circuit 13, the correction of the attenuation due to the propagation distance according to the depth of the position at which the ultrasound wave is reflected, and generates the signal that is the tomographic image information related to the tissue in the subject.

The first image processing unit 17 performs various types of processing on the signal generated by the first signal processing unit 16 to generate the first ultrasound image, such as the B mode image, the M mode image, the color Doppler image, or the pulsed Doppler image. Further, the first image processing unit 17 compresses the generated first ultrasound image into the format, such as JPEG, and sends out the generated first ultrasound image to the wireless communication circuit 19.

The wireless communication circuit 19 of the ultrasound probe 2 wirelessly transmits the first ultrasound image compressed in the format, such as JPEG, to the wireless communication circuit 41 of the mobile information terminal 4.

The wireless communication circuit 41 of the mobile information terminal 4 sends out the first ultrasound image transmitted from the wireless communication circuit 19 of the ultrasound probe 2 to the display control unit 42.

The first ultrasound image is displayed on the monitor 43 after being subjected to various types of processing by the display control unit 42.

In a state in which the first ultrasound image is displayed on the monitor 43, for example, the user can advance the ultrasound examination with respect to the subject by performing the input operation for freeze-displaying the first ultrasound image on the monitor 43 or the like via the input device 37 of the user interface device 3.

Here, although the mobile information terminal 4 comprises the input device 44 including the touch panel, it is necessary to get used to the input operation via the touch panel, and in particular, in a case in which a user having a low skill level performs the input operation, there is a case in which the ultrasound examination with respect to the subject cannot be smoothly performed. Since the input device 37 of the user interface device 3 may have input means, which is specialized for the ultrasound examination and includes the dial, the track ball, various buttons, and the like, the user can smoothly perform the ultrasound examination with respect to the subject.

In this way, the operation of the ultrasound system 1 in a case in which the first ultrasound image is displayed on the monitor 43 of the mobile information terminal 4 is completed.

Next, the operation of the ultrasound system 1 in a case in which the second ultrasound image is generated in the user interface device 3, and the second ultrasound image is displayed on the monitor 43 of the mobile information terminal 4 will be described.

First, the user inputs an instruction to generate the second ultrasound image via the input device 37 of the user interface device 3. In this case, for example, the input device 37 comprises a button for inputting the instruction to generate the second ultrasound image, and the user can press this button to input the instruction to generate the second ultrasound image.

Next, information indicating the instruction to generate the second ultrasound image is transmitted to the wired communication terminal 20 of the ultrasound probe 2 via the wired communication terminal 31 of the user interface device 3, and is transmitted from the wired communication terminal 20 to the probe control unit 21. The probe control unit 21 controls the transmission/reception circuit 14 via the ultrasound transmission/reception control unit 15 to cause the transducer array 11 to perform the transmission and the reception of the ultrasound wave for generating the second ultrasound image.

In a case in which the ultrasound beam is transmitted from the transducer array 11 into the subject, and the ultrasound echo is received by the reception circuit 13, the reception circuit 13 generates the sound ray signal, and sends out the generated sound ray signal to the wired communication terminal 20.

The sound ray signal sent out to the wired communication terminal 20 is transmitted to the wired communication terminal 31 of the user interface device 3 via the cable (not shown) that connects the wired communication terminal 20 of the ultrasound probe 2 and the wired communication terminal 31 of the user interface device 3 to each other, and is further sent out to the second signal processing unit 32.

The second signal processing unit 32 performs the quadrature detection processing and the envelope detection processing after performing, with respect to the sound ray signal sent out from the wired communication terminal 31, the correction of the attenuation due to the propagation distance according to the depth of the position at which the ultrasound wave is reflected, and generates the signal that is the tomographic image information related to the tissue in the subject.

The second image processing unit 33 performs various types of processing with respect to the signal generated by the second signal processing unit 32 to generate the second ultrasound images, such as the ultrasound elastography image, the low-speed blood flow image, the high-sensitivity low-speed image, and the contrast image. In order to generate such a second ultrasound image, processing that has a larger computing amount and is more complicated than the first ultrasound image generated by the first image generation unit 18 in the ultrasound probe 2 is required.

However, in general, in the ultrasound probe, it is necessary to suppress heat generation in the ultrasound probe in order to reduce a burden on the subject or the like, and thus it is difficult to mount a circuit on an ultrasound probe in which a heat generation amount is increased due to complicated processing. Further, in the ultrasound probe capable of wireless communication, since power is supplied from a battery provided inside the ultrasound probe, in a case of considering that the examination with respect to the subject is performed in a sufficiently stable manner, it is difficult to mount a circuit that performs processing with large power consumption in the ultrasound probe.

Therefore, in the ultrasound system that can generate the ultrasound image only in the ultrasound probe, it is difficult for the user to perform various examinations with respect to the subject, for example, to perform the examination with respect to the subject by generating a plurality of types of ultrasound images or the like.

With the second image generation unit 34 of the user interface device 3 in the first embodiment, the second ultrasound image, which requires complicated processing in a case of the generation, is generated, so that the user can perform various examinations with respect to the subject.

The second image processing unit 33 sends out the second ultrasound image to the wireless communication circuit 35.

The wireless communication circuit 35 of the user interface device 3 transmits the second ultrasound image to the wireless communication circuit 41 of the mobile information terminal 4.

The second ultrasound image received by the wireless communication circuit 41 of the mobile information terminal 4 is displayed on the monitor 43 after being subjected to various types of processing by the display control unit 42.

In a state in which the second ultrasound image is displayed on the monitor 43, for example, the user can advance the ultrasound examination with respect to the subject by performing the input operation for freeze-displaying the second ultrasound image on the monitor 43 or the like via the input device 37 of the user interface device 3.

In this way, the operation of the ultrasound system 1 in a case in which the second ultrasound image is displayed on the monitor 43 of the mobile information terminal 4 is completed.

From the above, with the ultrasound system 1 according to the first embodiment of the present invention, the input operation of the user is performed via the input device 37 of the user interface device 3, the first ultrasound image is generated by the first image generation unit 18 of the ultrasound probe 2, and the second ultrasound image, which requires complicated processing for the generation, is generated by the second image generation unit 34 of the user interface device 3, so that the user can easily perform various examinations with respect to the subject.

It should be noted that, although it is described that the user interface device 3 and the mobile information terminal 4 are connected to each other by wireless communication, the user interface device 3 and the mobile information terminal 4 can be connected to each other by wired connection.

In addition, in a case in which the first ultrasound image is generated by the ultrasound probe 2, the mobile information terminal 4 does not have to be connected to the user interface device 3. In this case, the user can perform the input operation via the input device 44 of the mobile information terminal 4.

In addition, it is described that the ultrasound probe 2 comprising the first image processing unit 17 is connected to the user interface device 3, but an ultrasound probe (not shown) that does not comprise the first image processing unit 17 may be connected. In this case, only the second ultrasound image is generated without generating the first ultrasound image. However, the user performs the input operation with respect to the ultrasound system 1 via the input device 37 of the user interface device 3, so that the user can smoothly advance the ultrasound examination with respect to the subject.

In addition, it is described that the second image processing unit 33 generates the second ultrasound images, such as the ultrasound elastography image, the low-speed blood flow image, the high-sensitivity low-speed image, and the contrast image, based on the signal generated by the second signal processing unit 32, but the second signal processing unit 32 can perform signal processing according to the type of the second ultrasound image. Also in this case, the second image generation unit 34 generates the second ultrasound image.

In addition, it is described that the user inputs the instruction to generate the second ultrasound image via the input device 37 of the user interface device 3, but the user can also select, for example, a mode of generating any one of the ultrasound elastography image, the low-speed blood flow image, the high-sensitivity low-speed image, or the contrast image as the second ultrasound image via the input device 37 in a case in which the ultrasound examination with respect to the subject is started. In this way, in a case in which the mode is selected by the user, the device control unit 36 can automatically transmit the instruction to generate the second ultrasound image to the ultrasound probe 2 via the wired communication terminal 31.

### Second Embodiment

Although the power is supplied to each unit of the ultrasound probe 2 from the built-in battery 22, it is also possible to supply the power to each unit of the ultrasound probe 2 from the user interface device 3.

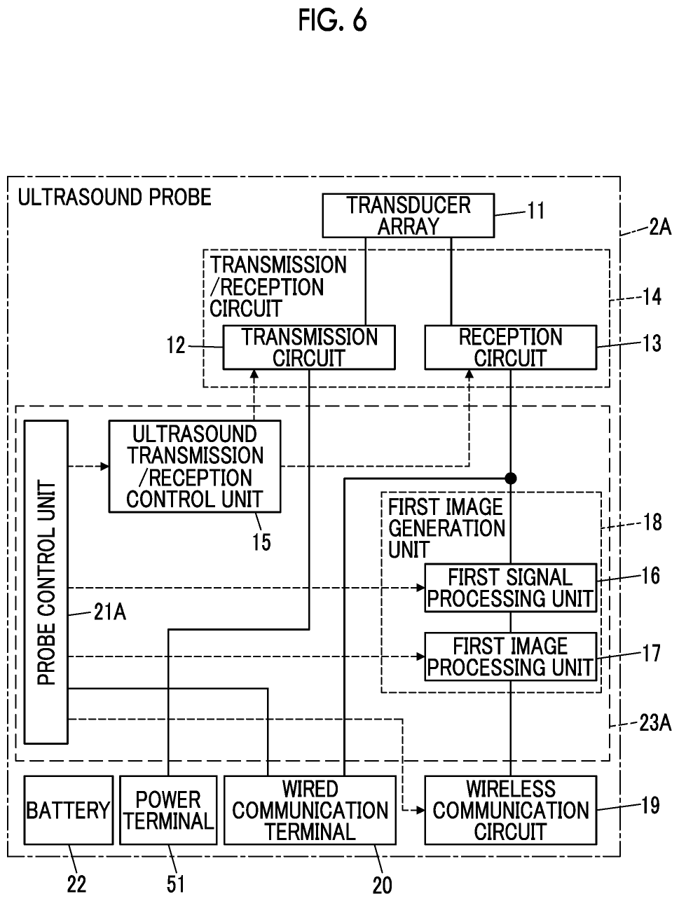

FIG. 6 shows a configuration of an ultrasound probe 2A in a second embodiment.

The ultrasound probe 2A in the second embodiment has a configuration in which a power terminal 51 is added, a probe control unit 21A is provided instead of the probe control unit 21, and an ultrasound probe processor 23A is provided instead of the ultrasound probe processor 23 in the ultrasound probe 2 in the first embodiment shown in FIG. 2.

In the ultrasound probe 2A, the power terminal 51 is connected to the transmission circuit 12. Also, although not shown, the power terminal 51 is connected to each unit of the ultrasound probe 2A.

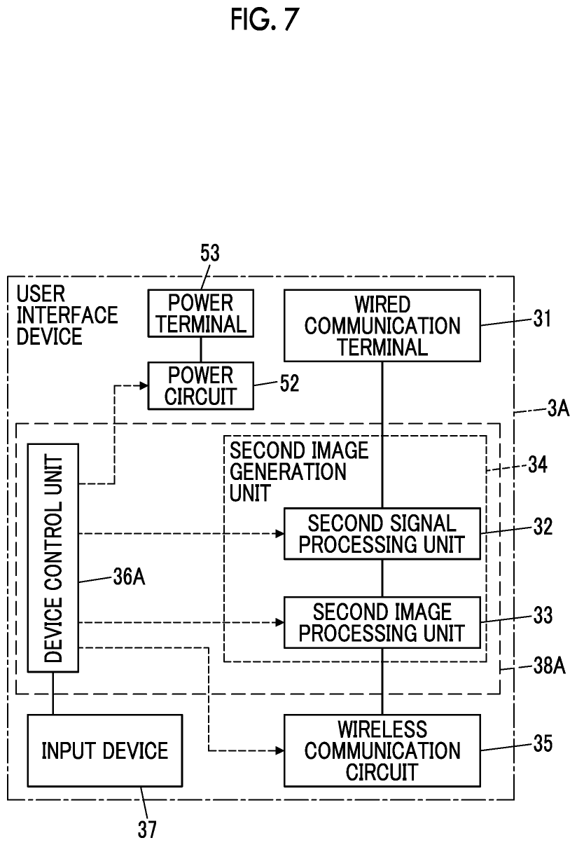

FIG. 7 shows a configuration of a user interface device 3A in the second embodiment.

The user interface device 3A in a second embodiment has a configuration in which a power circuit 52 and a power terminal 53 are added, a device control unit 36A is provided instead of the device control unit 36, and a user interface device processor 38A is provided instead of the user interface device processor 38 in the user interface device 3 shown in FIG. 4.

In the user interface device 3A, the device control unit 36A and the power terminal 53 are connected to the power circuit 52. Also, the power terminal 53 is connected to the power terminal 51 of the ultrasound probe 2A via the cable (not shown).

It should be noted that the cable that connects the power terminal 51 of the ultrasound probe 2A and the power terminal 53 of the user interface device 3A to each other can be put together with the cable (not shown) that connects the wired communication terminal of the ultrasound probe 2A and the wired communication terminal 31 of the user interface device 3A to each other.

The power circuit 52 of the user interface device 3A is for supplying power to the ultrasound probe 2A connected by wire. In this case, the voltage generated in the power circuit 52 is supplied to each unit of the ultrasound probe 2A via the power terminal 53, the cable (not shown), and the power terminal 51 of the ultrasound probe 2A.

In addition, the power circuit 52 can supply a transmission voltage for transmitting the ultrasound wave from the transducer array 11 of the ultrasound probe 2A to the transmission/reception circuit 14, particularly the transmission circuit 12.

From the above, with the ultrasound system in the second embodiment, the power is supplied to each unit of the ultrasound probe 2A from the power circuit 52 of the user interface device 3A. Therefore, the user can perform the examination with respect to the subject for a longer time than in a case in which the power is supplied to each unit of the ultrasound probe 2A from only the battery 22 built in the ultrasound probe 2A, and thus it is possible to sufficiently examine the subject.

In addition, the power circuit 52 of the user interface device 3A can supply the transmission voltage for transmitting the ultrasound wave from the transducer array 11 of the ultrasound probe 2A, to the transmission/reception circuit 14, and thus it is possible to obtain a high-definition ultrasound image by increasing the intensity of the ultrasound wave transmitted into the subject.

Also, in this case, the ultrasound transmission/reception control unit 15 can control a level of the voltage applied to the transducer array 11 to, for example, apply a complex transmission waveform, such as a coded transmission waveform, to the transducer array 11.

### Third Embodiment

Although it is described that one ultrasound probe 2 is connected to the user interface device 3 in the first embodiment, a plurality of ultrasound probes 2 can be connected to the user interface device 3.

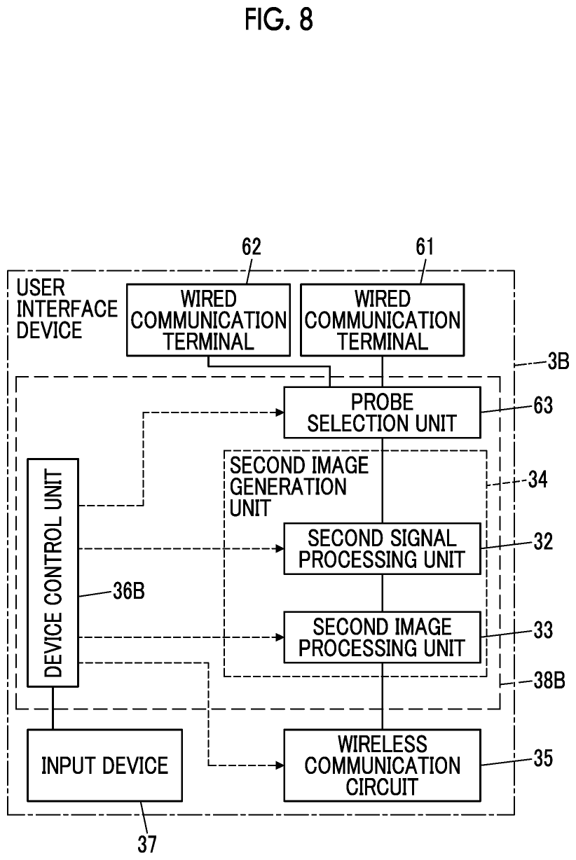

FIG. 8 shows a configuration of a user interface device 3B in the third embodiment.

The user interface device 3B has a configuration in which two wired communication terminals 61 and 62 are provided instead of one wired communication terminal 31, a probe selection unit 63 is added, a device control unit 36B is provided instead of the device control unit 36, and a user interface device processor 38B is provided instead of the user interface device processor 38 in the user interface device 3 in the first embodiment shown in FIG. 4.

In the user interface device 3B, the probe selection unit 63 is connected to the wired communication terminal 61 and the wired communication terminal 62. In addition, the second signal processing unit 32 and the device control unit 36B are connected to the probe selection unit 63.

In addition, the user interface device processor 38B is configured by using the second signal processing unit 32, the second image processing unit 33, the device control unit 36B, and the probe selection unit 63.

The wired communication terminal 61 and the wired communication terminal 62 are the same as the wired communication terminal 31 in the first embodiment. The ultrasound probes (not shown) that are different from each other are connected to the wired communication terminal 61 and the wired communication terminal 62, respectively. These ultrasound probes have the same configuration as the ultrasound probe 2 in the first embodiment.

The probe selection unit 63 selects one ultrasound probe of two ultrasound probes connected to the wired communication terminal 61 and the wired communication terminal 62. The probe selection unit 63 can select one ultrasound probe based on, for example, the input operation performed by the user via the input device 37 of the user interface device 3B.

The probe selection unit 63 sends out the sound ray signal generated by the selected ultrasound probe to the second signal processing unit 32.

As a result, the second image generation unit 34 generates the second ultrasound image based on the sound ray signal generated by the transmission/reception circuit of the ultrasound probe selected by the probe selection unit 63.

From the above, with the ultrasound system according to the third embodiment, two ultrasound probes are connected to the user interface device 3B, and one ultrasound probe to be used for the generation of the second ultrasound image is selected by the probe selection unit 63. Therefore, the user can perform the examination with respect to the subject while easily changing the type of the ultrasound probe according to the type of the examination, the examination location, and the like.

It should be noted that, although the example is described in which the user interface device 3B comprises the two wired communication terminals 61 and 62, the user interface device 3B can comprise a plurality (three or more) of wired communication terminals. In this case, since the user can easily use more types of ultrasound probes, the user can perform more various examinations with respect to the subject.

In addition, the user interface device 3B can also comprise the reception circuit 13 shown in FIG. 3. In this case, the reception circuit 13 is, for example, connected to the device control unit 36B and the probe selection unit 63, and is further connected to the second signal processing unit 32. In a case in which the user interface device 3B comprises the reception circuit 13, the ultrasound probe that does not comprise the reception circuit 13 can also be connected to the user interface device 3B.

In addition, in this case, the user interface device 3B can also comprise the transmission circuit 12 connected in parallel to the reception circuit 13 to the probe selection unit 63.

In addition, it is described that the wired communication terminal 62 is the same as the wired communication terminal 31 in the first embodiment. However, for example, the wired communication terminal 62 may be configured by using a plurality of terminals corresponding to the number of ultrasound transducers provided in the transducer array 11, for an ultrasound probe (not shown) configured by using the transducer array 11. In a case in which the wired communication terminal 62 is configured for the ultrasound probe (not shown) configured by using the transducer array 11 and the user interface device 3B comprises the transmission circuit 12 and the reception circuit 13, in a case in which the ultrasound probe (not shown) configured by using the transducer array 11 is connected to the wired communication terminal 62, the probe selection unit 63 may automatically connect the wired communication terminal 62 to the transmission circuit 12 and the reception circuit 13.

In addition, although it is described that the aspect of the third embodiment can be applied to the first embodiment, the aspect can be applied to the second embodiment in the same manner.

### Fourth Embodiment

Although, in the first embodiment, it is described that the second image generation unit 34 can generate the ultrasound elastography image, the low-speed blood flow image, the high-sensitivity low-speed image, and the contrast image as the second ultrasound images, but it is also possible to generate a continuous wave Doppler image.

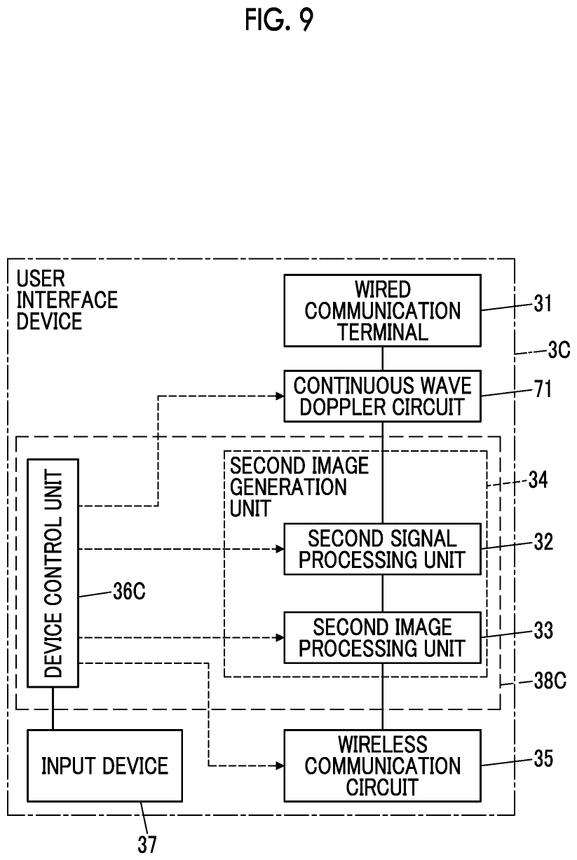

FIG. 9 shows a configuration of a user interface device 3C in a fourth embodiment.

The user interface device 3C has a configuration in which a continuous wave Doppler circuit 71 is added, the device control unit 36 is provided instead of the device control unit 36C, and a user interface device processor 38C is provided instead of the user interface device processor 38 in the user interface device 3 in the first embodiment shown in FIG. 4.

In the user interface device 3C, the continuous wave Doppler circuit 71 is connected to the wired communication terminal 31. In addition, the second signal processing unit 32 and the device control unit 36C are connected to the continuous wave Doppler circuit 71.

The ultrasound probe (not shown) that can operate in a continuous wave Doppler mode, that is, that can perform the transmission and the reception of the ultrasound wave for generating the continuous wave Doppler image can be connected to the wired communication terminal 31. In this case, the reception signal acquired by the transducer array is transmitted from the ultrasound probe to the user interface device 3C via the wired communication terminal 31.

In a case in which the ultrasound probe that can operate in the continuous wave Doppler mode is connected to the wired communication terminal 31, the continuous wave Doppler circuit 71 drives the transducer array of the ultrasound probe in the continuous wave Doppler mode and generates the sound ray signal based on the reception signal acquired by the transducer array.

In addition, the continuous wave Doppler circuit 71 sends out the generated sound ray signal to the second signal processing unit 32.

As a result, the second image generation unit 34 generates the continuous wave Doppler image based on the sound ray signal generated by the continuous wave Doppler circuit 71. The continuous wave Doppler image generated in this way is an image generated by more complicated processing than in the generation of the first ultrasound image, similar to the ultrasound elastography image, the low-speed blood flow image, the high-sensitivity low-speed image, and the contrast image.

From the above, with the ultrasound system according to the fourth embodiment, the continuous wave Doppler image can be generated as the second ultrasound image, so that the user can perform more various examinations with respect to the subject.

It should be noted that, although it is described that the aspect of the fourth embodiment can be applied to the first embodiment, the aspect can be applied to the second embodiment and the third embodiment in the same manner.

### EXPLANATION OF REFERENCES

1: ultrasound system
2, 2A: ultrasound probe
3, 3A, 3B, 3C: user interface device
4: mobile information terminal
11: transducer array
12: transmission circuit
13: reception circuit
14: transmission/reception circuit
15: ultrasound transmission/reception control unit
16: first signal processing unit
17: first image processing unit
18: first image generation unit
19, 35, 41: wireless communication circuit
31, 61, 62: wired communication terminal
21, 21A: probe control unit
22: battery
23, 23A: ultrasound probe processor
24: amplification unit
25: AD conversion unit
26: beam former
32: second signal processing unit
33: second image processing unit
34: second image generation unit
36, 36A, 36B, 36C: device control unit
37, 44: input device
38, 38A, 38B, 38C: user interface device processor
42: display control unit
43: monitor
45: terminal control unit
46: mobile information terminal processor
51, 53: power terminal
52: power circuit
63: probe selection unit
71: continuous wave Doppler circuit

What is claimed is:

1. An ultrasound system comprising:
an ultrasound probe;
a user interface device connected to the ultrasound probe by wire; and
a mobile information terminal connected to at least one of the ultrasound probe or the user interface device,
wherein the ultrasound probe includes
a transducer array,
a transmission circuit configured to transmit an ultrasound wave from the transducer array,
a reception circuit configured to generate a sound ray signal based on a reception signal acquired by the transducer array, and
a first processor configured to generate a first ultrasound image based on the sound ray signal generated by the reception circuit,
the user interface device includes
a second processor configured to generate a second ultrasound image based on the sound ray signal generated by the reception circuit of the ultrasound probe, and
the mobile information terminal includes
a monitor configured to display the first ultrasound image generated by the first processor of the ultrasound probe or the second ultrasound image generated by the second processor of the user interface device.

2. The ultrasound system according to claim 1,
wherein the second ultrasound image generated by the second processor of the user interface device includes at least one of an ultrasound elastography image, a low-speed blood flow image, a high-sensitivity low-speed image, or a contrast image.

3. The ultrasound system according to claim 1,
wherein the mobile information terminal is configured to accept an input operation of a user in a case in which the mobile information terminal is not connected to the user interface device.

4. The ultrasound system according to claim 2,
wherein the mobile information terminal is configured to accept an input operation of a user in a case in which the mobile information terminal is not connected to the user interface device.

5. The ultrasound system according to claim 1,
wherein the mobile information terminal is wirelessly connected to the ultrasound probe, and
the first ultrasound image generated by the first processor of the ultrasound probe is wirelessly transmitted from the ultrasound probe to the mobile information terminal and displayed on the monitor.

6. The ultrasound system according to claim 2,
wherein the mobile information terminal is wirelessly connected to the ultrasound probe, and
the first ultrasound image generated by the first processor of the ultrasound probe is wirelessly transmitted from the ultrasound probe to the mobile information terminal and displayed on the monitor.

7. The ultrasound system according to claim 3,
wherein the mobile information terminal is wirelessly connected to the ultrasound probe, and
the first ultrasound image generated by the first processor of the ultrasound probe is wirelessly transmitted from the ultrasound probe to the mobile information terminal and displayed on the monitor.

8. The ultrasound system according to claim 1,
wherein the user interface device includes a power circuit configured to supply power to the ultrasound probe connected to the user interface device by wire.

9. The ultrasound system according to claim 2,
wherein the user interface device includes a power circuit configured to supply power to the ultrasound probe connected to the user interface device by wire.

10. The ultrasound system according to claim 3,
wherein the user interface device includes a power circuit configured to supply power to the ultrasound probe connected to the user interface device by wire.

11. The ultrasound system according to claim 5,
wherein the user interface device includes a power circuit configured to supply power to the ultrasound probe connected to the user interface device by wire.

12. The ultrasound system according to claim 8,
wherein the power circuit of the user interface device is further configured to supply a transmission voltage for transmitting the ultrasound wave from the transducer array of the ultrasound probe, to the transmission circuit.

13. The ultrasound system according to claim 1,
wherein each of a plurality of the ultrasound probes is connected to the user interface device by wire,
the second processor of the user interface device is further configured to
select one ultrasound probe among the plurality of ultrasound probes, and
generate the second ultrasound image based on the sound ray signal generated by the reception circuit of the ultrasound probe which is selected.

14. The ultrasound system according to claim 2,
wherein each of a plurality of the ultrasound probes is connected to the user interface device by wire,
the second processor of the user interface device is further configured to
select one ultrasound probe among the plurality of ultrasound probes, and
generate the second ultrasound image based on the sound ray signal generated by the reception circuit of the ultrasound probe which is selected.

15. The ultrasound system according to claim 3,
wherein each of a plurality of the ultrasound probes is connected to the user interface device by wire,
the second processor of the user interface device is further configured to
select one ultrasound probe among the plurality of ultrasound probes, and
generate the second ultrasound image based on the sound ray signal generated by the reception circuit of the ultrasound probe which is selected.

16. The ultrasound system according to claim 5,
wherein each of a plurality of the ultrasound probes is connected to the user interface device by wire,
the second processor of the user interface device is further configured to
select one ultrasound probe among the plurality of ultrasound probes, and
generate the second ultrasound image based on the sound ray signal generated by the reception circuit of the ultrasound probe which is selected.

17. The ultrasound system according to claim 12,
wherein each of a plurality of the ultrasound probes is connected to the user interface device by wire,
the second processor of the user interface device is further configured to
select one ultrasound probe among the plurality of ultrasound probes, and
generate the second ultrasound image based on the sound ray signal generated by the reception circuit of the ultrasound probe which is selected.

18. The ultrasound system according to claim 1,
wherein the user interface device includes a continuous wave Doppler circuit configured to drive the transducer array in a continuous wave Doppler mode and generate a sound ray signal based on the reception signal acquired by the transducer array, and
the second processor configured to generate a continuous wave Doppler image based on the sound ray signal generated by the continuous wave Doppler circuit.

19. The ultrasound system according to claim 2,
wherein the user interface device includes a continuous wave Doppler circuit configured to drive the transducer array in a continuous wave Doppler mode and generate a sound ray signal based on the reception signal acquired by the transducer array, and
the second processor configured to generate a continuous wave Doppler image based on the sound ray signal generated by the continuous wave Doppler circuit.

20. A control method of an ultrasound system including an ultrasound probe, a user interface device connected to the ultrasound probe by wire, and a mobile information terminal connected to at least one of the ultrasound probe or the user interface device, the control method comprising:
causing the ultrasound probe
to transmit an ultrasound wave from a transducer array,
to generate a sound ray signal based on a reception signal acquired by the transducer array, and
to generate a first ultrasound image based on the sound ray signal;
causing the user interface device
to generate a second ultrasound image based on the sound ray signal; and
causing the mobile information terminal
to display the first ultrasound image or the second ultrasound image on a monitor.
